(19) United States
(12) Patent Application Publication
(10) Pub. No.: US 2006/0228312 A1
(43) Pub. Date: Oct. 12, 2006

(54) Delivery system for film-forming polymer

(75) Inventors: David H. Weiss, Maplewood, NJ (US); Steven W. Rogers, Yardley, PA (US)

Correspondence Address: CROWELL & MORING LLP;INTELLECTUAL PROPERTY GROUP, P.O. BOX 14300, WASHINGTON, DC, 20044-4300, US

(73) Assignee: Rutherford Chemicals LLC - CasChem Unit

(21) Appl. No.: 11/102,223

(22) Filed: Apr. 8, 2005

(51) Int. Cl. A61K 8/84

(52) U.S. Cl. 424/59; 424/70.11

(57) ABSTRACT

A film-forming composition formed by solubilizing higher concentrations of polyanhydride resins using a mixture of a fatty acid triglyceride derivative and a diester of 2-Octanol and Adipic Acid. In one embodiment, a composition in accordance with the invention comprises between about 1% and 55% by weight of Furandione (2,5)-Polymer with 1-Octadecene; between about 10% and 35% by weight of Dicapryl Adipate; and between about 10% and 35% by weight of Methyl Acetyl Ricinoleate. Such a composition may impart a water resistant property to oil-in-water and water-in-oil emulsions, and to anhydrous solid and semi-solid preparations, while remaining solubilized, stable and pourable at 25° C.

### FIELD OF THE INVENTION

This invention relates to film-forming compositions and, in particular, to film-forming compositions used to impart water resistance properties in sunscreen and cosmetic applications.

### BACKGROUND OF THE INVENTION

A water resistance agent is a hydrophobic material that imparts film forming characteristics to an emulsion. Such waterproofing agents have found application in the cosmetic and sunscreen industries for years. Various water resistance agents used include copolymers of vinyl pyrollidone and eicosene and dodecane monomers, polyurethane polymer, polyethylene and polyanhydride resin.

It has been found that increasing the relative concentration of the film-forming composition also increases the water resistance and anti-transfer properties of the resulting product. Most polymeric film-forming agents used for water resistance in cosmetic formulations are high molecular weight solids which are have low solubilities in these formulations. These polymeric film-forming agents are most easily incorporated when they are pre-dissolved or dispersed in solvents which maintain the highest concentration of the polymeric film forming agents, thus minimizing the amount of solvent carried into the formulation. Presently-used solvents are incapable of achieving high solubilization rates without causing the film-forming composition to become unstable and difficult to pour. Low concentrations of film-forming compositions are also undesirable since a higher relative concentration of the solvent will be carried in the end product—such solvents often impart undesirable properties in the final product.

By way of example, one approach to producing a water resistant or film-forming agent has been to create a solution of a linear polyanhydride resin in a diester that is the reaction product of 2-ethylhexanol and sebacic acid prepared having a concentration of 20% w/w and a viscosity of 108 cP. At this concentration, the composition is a transparent, pourable liquid that is free of entrapped air and particulate matter. However, increasing the concentration to even 40% w/w changes the composition's physical form to an opaque semi-solid gel having a viscosity greater than 50,000 cP. In this form, the composition is unstable (i.e., separates and crystallizes) and is very difficult to handle and dispense.

Similarly, other solvents, alone or in combination, that find use in cosmetic oil in water or water in oil emulsions include C12-15 Alkyl Benzoate, Propylene Glycol Ricinoleate, Ethyl Hexyl Palmitate and 2-Propenoic Acid, 2-Cyano-3,3-Diphenyl-, 2-Ethylhexyl Ester. However, such solvents form difficult to dispense semi-solids with viscosities greater than 50,000 cP at relatively low concentrations, thereby increasing the relative amounts of solvents being carried in the final product. For example, above a concentration of about 35% w/w, a resin solubilized with C12-15 Alkyl Benzoate becomes unusable as a viable delivery system. For 2-Propenoic Acid, 2-Cyano-3,3-Diphenyl-, 2-Ethylhexyl Ester, the maximum concentration is only about 20% w/w. And the combination of Diethylhexyl Sebecate and C12-15 Alkyl Benzoate will produce a resin concentration of only about 33% before becoming an effective delivery system.

Thus, there is a need for a film-forming agent and method of forming the same which overcomes one or more of the aforementioned drawbacks.

### SUMMARY OF THE INVENTION

A film-forming composition comprising from about 10% to about 35% by weight of Methyl Acetyl Ricinoleate, from about 10% to about 35% by weight of Dicapryl Adipate, and from about 1% to about 55% by weight of a polyanhydride resin. In another embodiment, the film-forming composition comprising from about 20% to about 55% by weight of said polyanhydride resin.

Other aspects, features, and techniques of the invention will be apparent to one skilled in the relevant art in view of the following detailed description of the invention.

### BRIEF DESCRIPTION OF THE DRAWINGS

FIG. 1 is a molecular representation of Furandione (2,5)-Polymer with 1-Octadecene;

FIG. 2 is a molecular representation of Methyl Acetyl Ricinoleate; and

FIG. 3 is a molecular representation of Dicapryl Adipate.

### DETAILED DESCRIPTION OF THE EXEMPLARY EMBODIMENTS

Polyanhydride resins provide excellent water resistance and film-forming properties for use in both sunscreen and cosmetic applications. As a general rule, the higher the concentration of the resin the better the water resistance and film-forming properties provided. The resins are best added to formulations when pre-dissolved or pre-dispersed. However, at higher concentrations polyanhydride resins become unstable opaque semi-solid gels which are difficult to work with. To that end, one aspect of the invention is to be able to solubilize higher concentrations of polyanhydride resins using a mixture of a fatty acid triglyceride derivative and a diester of 2-Octanol (Capryl Alcohol) and Adipic Acid to produce a stable, easy-to-use water resistant composition that is pourable at 25° C. In one embodiment, the composition may also be transparent to translucent.

In one embodiment, a composition consistent with the principles of the invention contains a high concentration of a Poly Maleic Anhydride-Alt-1-Octadecene in a mixture of a fatty acid triglyceride derivative and a diester of 2-Octanol (Capryl Alcohol) and Adipic Acid. Such a composition may be usable to impart a water resistant or very water resistant property to oil-in-water and water-in-oil emulsions. In one embodiment, the composition comprises between about 1% and 55% by weight of Furandione (2,5)-Polymer with 1-Octadecene; between about 10% and 35% by weight of Dicapryl Adipate; and between about 10% and 35% by weight of Methyl Acetyl Ricinoleate. In another embodiment, the composition comprises between about 20% and 55% by weight of Furandione (2,5)-Polymer with 1-Octadecene, with the remaining components including Dicapryl Adipate and Methyl Acetyl Ricinoleate. In still a third embodiment, the composition comprises about 50% by weight of Furandione (2,5)-Polymer with 1-Octadecene, about 25% by weight of Dicapryl Adipate, and about 25% by weight of Methyl Acetyl Ricinoleate.

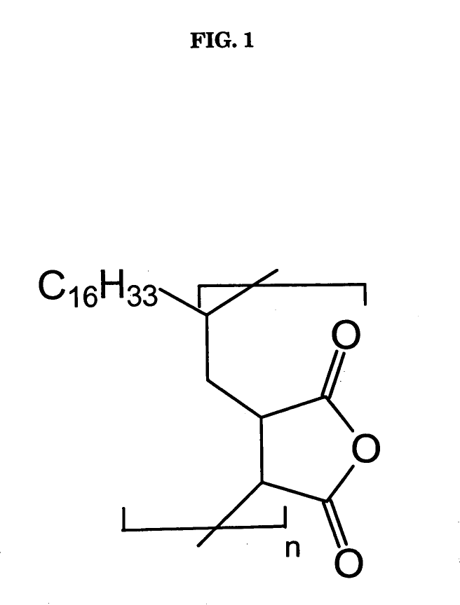

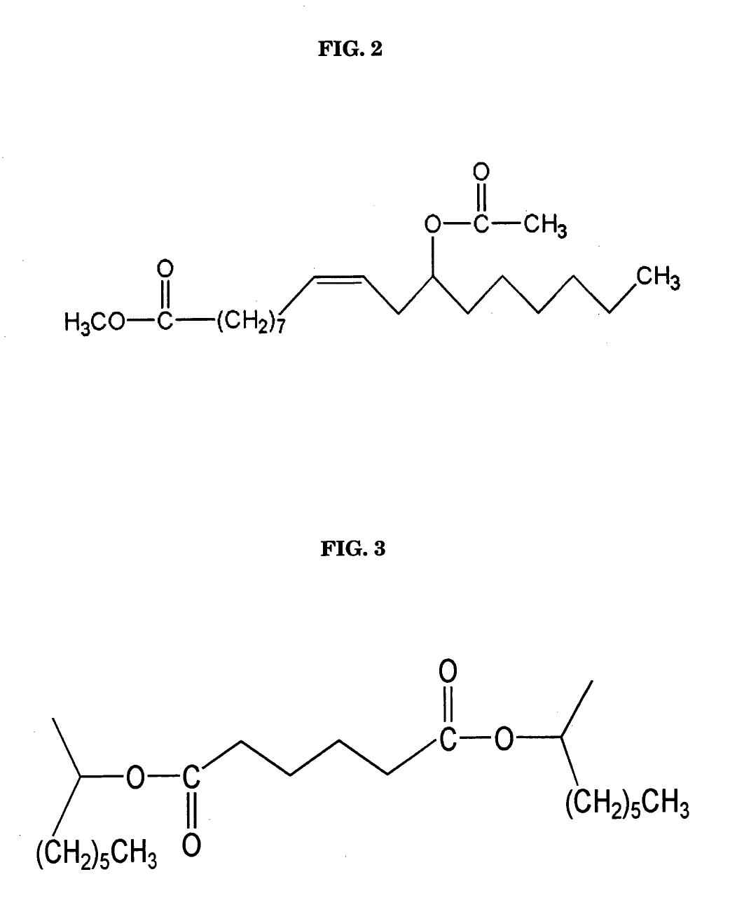

FIG. 1 represents a molecule of Furandione (2,5)-Polymer with 1-Octadecene. Similarly, FIG. 2 represents a molecule of Methyl Acetyl Ricinoleate, while FIG. 3 represents a molecule of Dicapryl Adipate.

In one embodiment, the Dicapryl Adipate (having the INCI name of Diisooctyl Adipate) to be used in the above mixture is the product sold under the tradename Casmate® DCA by Rutherford Chemicals LLC CasChem. Similarly, the Methyl Acetyl Ricinoleate to be used may be the product sold under the tradename Naturechem® MAR also by Rutherford Chemicals LLC CasChem. Finally, one polyanhydride resin which may be used is the product manufactured and sold under the name PA-18 HVLC Polyanhydride Resin by the Chevron Philips Chemical Company LP.

Another aspect of the invention is to use the aforementioned composition as a cosmetic emollient to retard the rate of transepidermal water loss by forming an occlusive barrier on the epidermis. In one embodiment, a mixture of Dicapryl Adipate and Methyl Acetyl Ricinoleate is used to solubilize a linear polyanhydride resin (e.g., MW=40,000-50,000) at a w/w concentration of between 20% and 55% to form a viscous, pourable, transparent (or translucent) liquid at 25° C. Such a composition may provide a workable, dispensable and emollient vehicle for the delivery of a cosmetic or sunscreen ingredient. In another embodiment, such a composition may be capable of imparting a water resistant or very water resistant property to oil-in-water and water-in-oil emulsions and to anhydrous solid and semi-solid preparations. In still another embodiment, a composition prepared in accordance with the principles of the invention may retard the rate of transepidermal water loss by forming an occlusive film on the epidermal surface, while also being pourable at 25° C.

### EXAMPLE 1

Using a steam jacketed stainless steel mixing tank equipped with a stainless steel propeller mixer and cover, 25 kg of Dicapryl Adipate was added at 25° C. Slow to moderate mixing was then used while adding 25 kg of Methyl Acetyl Ricinoleate. This mixture was then heated to a target temperature of 120° C. at a rate of approximately 2° C. per minute. During this heating period, 50 kg of PA-18 HVLC Polyanhydride Resin was slowly and incrementally added. Once all of the PA-18 HVLC Polyanhydride Resin was added, the mixing speed was increased to accommodate the dispersion of the resin powder.

Once all of the resin powder has been dispersed, the mixing speed was reduced to avoid aerating the mixture and mixing was continued until the batch reached 120° C. This temperature was maintained for 30 minutes while slow mixing was continued. Heating was then stopped and the batch temperature was decreased to 50° C. with continued slow mixing under vacuum conditions.

### EXAMPLE 2

Using the same mixing tank and conditions as with Example 1, 10 kg of Dicapryl Adipate was added at 25° C. Slow to moderate mixing was then used while adding 35 kg of Methyl Acetyl Ricinoleate. This mixture was then slowly heated to a target temperature of 120° C. During this heating period, 55 kg of PA-18 HVLC Polyanhydride Resin. As before, the mixing speed was increased to accommodate the dispersion of the resin powder. Once the of the resin powder has been dispersed, the mixing speed was reduced and heating continued until the batch reached 120° C. This temperature was maintained until all solids were melted and dispersed, while slow mixing was continued.

### EXAMPLE 3

Using the same mixing tank and conditions as with the previous examples, 35 kg of Dicapryl Adipate was mixed with 35 kg of Methyl Acetyl Ricinoleate at 25° C. This mixture was then slowly heated to a target temperature of 120° C., during which 30 kg of PA-18 HVLC Polyanhydride Resin was added. As before, the mixing speed was increased to accommodate the dispersion of the resin powder. Once the of the resin powder has been dispersed, the mixing speed was reduced and heating continued until the batch reached 120° C. This temperature was maintained until all solids were melted and dispersed, while slow mixing was continued.

### EXAMPLE 4

Using the same mixing tank and conditions as with the previous examples, 35 kg of Dicapryl Adipate was mixed with 10 kg of Methyl Acetyl Ricinoleate at 25° C. This mixture was then slowly heated to a target temperature of 120° C., during which 55 kg of PA-18 HVLC Polyanhydride Resin was added. As before, the mixing speed was increased to accommodate the dispersion of the resin powder. Once the of the resin powder has been dispersed, the mixing speed was reduced and heating continued until the batch reached 120° C. This temperature was maintained until all solids were melted and dispersed, while slow mixing was continued.

While the invention has been described in connection with various embodiments, it will be understood that the invention is capable of further modifications. This application is intended to cover any variations, uses or adaptation of the invention following, in general, the principles of the invention, and including such departures from the present disclosure as come within the known and customary practice within the art to which the invention pertains.

What is claimed is:

1. A film-forming composition comprising:
from about 10% to about 35% by weight of Methyl Acetyl Ricinoleate;
from about 10% to about 35% by weight of Dicapryl Adipate; and
from about 1% to about 55% by weight of a polyanhydride resin.

2. The film-forming composition of claim 1, comprising from about 20% to about 55% by weight of said polyanhydride resin.

3. The film-forming composition of claim 2, wherein said film-forming composition is completely solubilized and stable, transparent-to-translucent, pourable liquid at 25° C.

4. The film-forming composition of claim 3, wherein said film-forming composition has a viscosity of between 30,000 and 50,000 cP.

5. The film-forming composition of claim 1, wherein said polyanhydride resin is a solid linear polyanhydride resin derived from 1-Octadecene and Maleic Anhydride in a 1:1 molar ratio.

6. The film-forming composition of claim 1, wherein said polyanhydride resin has a molecular weight of between 40,000 and 50,000.

7. The film-forming composition of claim 1, wherein said polyanhydride resin is Furandione (2,5)-Polymer with 1-Octadecene.

8. The film-forming composition of claim 1, wherein said film-forming composition is usable to impart water resistant properties to sunscreens and anti-transfer cosmetics.

9. The film-forming composition of claim 1, wherein said film-forming composition is to retard a rate of transepidermal water loss by forming an occlusive film on an epidermal surface.

10. The film-forming composition of claim 1, wherein said film-forming composition is used in an anti-transfer makeup application.

11. The film-forming composition of claim 1, comprising:
about 25% by weight of Methyl Acetyl Ricinoleate;
about 25% by weight of Dicapryl Adipate; and
about 50% by weight of a polyanhydride resin.

12. A cosmetic emollient comprising:
from about 10% to about 35% by weight of Methyl Acetyl Ricinoleate;
from about 10% to about 35% by weight of Dicapryl Adipate; and
from about 1% to about 55% by weight of a polyanhydride resin.

13. The cosmetic emollient of claim 12, comprising from about 20% to about 55% by weight of said polyanhydride resin.

14. The cosmetic emollient of claim 13, wherein said cosmetic emollient is a solubilized and stable, transparent-to-translucent, pourable liquid at 25° C.

15. The cosmetic emollient of claim 14, wherein said cosmetic emollient has a viscosity of between 30,000 and 50,000 cP.

16. The cosmetic emollient of claim 12, wherein said polyanhydride resin is a solid linear polyanhydride resin derived from 1-Octadecene and Maleic Anhydride in a 1:1 molar ratio.

17. The cosmetic emollient of claim 12, wherein said polyanhydride resin has a molecular weight of between 40,000 and 50,000.

18. The cosmetic emollient of claim 12, wherein said film-forming composition is usable to impart water resistant properties to sunscreens and anti-transfer cosmetics.

19. The cosmetic emollient of claim 12, wherein said cosmetic emollient retards a rate of transepidermal water loss by forming an occlusive film on an epidermal surface.

20. The cosmetic emollient of claim 12, comprising:
about 25% by weight of Methyl Acetyl Ricinoleate;
about 25% by weight of Dicapryl Adipate; and
about 50% by weight of a polyanhydride resin.

21. The film-forming composition of claim 12, wherein said film-forming composition is used in an anti-transfer makeup application.

22. A method of producing a water resistance agent comprising:
mixing about 10% to about 35% by weight of Methyl Acetyl Ricinoleate with about 10% to about 35% by weight of Dicapryl Adipate to form a solubilizing agent;
heating said solubilizing agent; and
adding about 1% to about 55% by weight of a polyanhydride resin to said solubilizing agent to form said water resistance agent.

23. The method of claim 22, wherein adding comprises adding about 20% to about 55% by weight of said polyanhydride resin to said solubilizing agent.

24. The method of claim 23, wherein said water resistance agent is a solubilized and stable, transparent-to-translucent, pourable liquid at 25° C.

25. The method of claim 24, wherein said water resistance agent has a viscosity of between 30,000 and 50,000 cP.

26. The method of claim 22, wherein said polyanhydride resin is a solid linear polyanhydride resin derived from 1-Octadecene and Maleic Anhydride in a 1:1 molar ratio.

27. The method of claim 22, wherein said polyanhydride resin has a molecular weight of between 40,000 and 50,000.

28. The method of claim 22, wherein said polyanhydride resin is Furandione (2,5)-Polymer with 1-Octadecene.

29. The method of claim 22, further comprising adding said water resistance agent to at least one of a sunscreen product and an anti-transfer cosmetic product.

30. The method of claim 22, further comprising forming an occlusive film on an epidermal surface using said water resistance agent.

31. The method of claim 22, wherein mixing comprising mixing about 25% by weight of Methyl Acetyl Ricinoleate with about 25% by weight of Dicapryl Adipate to form a solubilizing agent, and wherein adding comprising adding about 50% by weight of the polyanhydride resin to said solubilizing agent to form said water resistance agent.

32. The method of claim 22, further comprising using said water resistance agent in an anti-trasfer makeup application.
